(19) United States
(12) Patent Application Publication
(10) Pub. No.: US 2026/0363538 A1
(43) Pub. Date: Nov. 19, 2026

(54) A METHOD OF USING OPTICAL COHERENCE TOMOGRAPHY FOR TRAINING A MACHINE LEARNING MODEL, A METHOD OF DETERMINING AN UNKNOWN CONDITION USING THE TRAINED MODEL, A METHOD OF DETERMINING SEGMENTATION IN A SAMPLE, AND AN OPTICAL COHERENCE TOMOGRAPHY SYSTEM

(71) Applicant: DANMARKS TEKNISKE UNIVERSITET, Kongens Lyngby (DK)

(72) Inventors: Peter E. ANDERSEN, Kongens Lyngby (DK); Gavrielle UNTRACHT, Kongens Lyngby (DK)

(21) Appl. No.: 19/138,752

(22) PCT Filed: Dec. 18, 2023
(86) PCT No.: PCT/EP2023/086303

§ 371 Date: Jun. 13, 2025

(30) Foreign Application Priority Data
Dec. 16, 2022 (EP) 22214290.3

(51) Int. Cl. A61B 3/10; G06N 3/08; G06V 10/141; G16H 30/20

(52) U.S. Cl. CPC A61B 3/102; G06N 3/08; G06V 10/141; G16H 30/20

(57) ABSTRACT

A method of using optical coherence tomography for training a machine learning model includes the steps of illuminating a first sample having a known condition along an illumination path, capturing a first training image of the sample along a first training collection path which is spatially and/or angularly offset from the illumination path by a first training offset, capturing a second training image of the sample along a second training collection path different from the first training collection path for providing a reference image, and training a machine learning model based on the first and second training images and the known condition.

### CROSS-REFERENCE TO RELATED APPLICATIONS

The present application is the national phase entry of International Patent Application No. PCT/EP2023/086303, filed Dec. 18, 2023, which claims priority to European Patent Application Ser. No. 22214290.3, filed Dec. 16, 2022, the entire contents of both are hereby incorporated by reference.

### FIELD OF DISCLOSURE

The disclosure relates to a method of using optical coherence tomography for training a machine learning model.

### BACKGROUND OF THE DISCLOSURE

Morphological imaging with optical coherence tomography (OCT) has made major strides in the past few decades, making rapid advances into the field of retinal imaging, revolutionizing applications in ophthalmology, and expanding into other fields including dermatology, cardiology, and the early detection of cancer. It is also used in numerous areas outside of biomedicine, such as art conservation and non-destructive testing. OCT relies on the coherence of light backscattered from the sample, with interference playing the important role of gating, allowing an accurate determination of the spatial origin of the retrieved signal. Backscattering occurs when there is a refractive index mismatch, typically generated by sub-resolution scatterers present within the sample microstructure. While advances of using OCT have been spectacular, there are major drawbacks in terms of its depth penetration. The state-of-the-art implementations of OCT use light in the near-infrared range of the spectrum to penetrate deeper into biological matter, however, signal extinction through turbid samples often precludes obtaining discernible signals from depths beyond 1 mm. The conventional wisdom is that the OCT signal is dominated by ballistically scattered light (light which has undergone a single backscattering event) whereas multiply scattered light and diffuse light are detrimental to image formation.

For that reason, the majority of approaches to date have aimed to minimize multiple scattering,

Cancer Res 79 (8), 2021-30 (2019) and US 2020/0359887 A1 disclose how to detect skin cancer (melanoma) using a combination of OCT, optical properties and machine learning.

### SUMMARY OF THE DISCLOSURE

Considering the prior art described above, it is an object of the present disclosure to use OCT to form images beyond depths corresponding to several scattering mean free paths, which in biological tissue typically corresponds to depths beyond 1 mm.

The object can be achieved in a first aspect by means of a method according to claim 1.

Using this method a machine learning model can be trained to recognize or provide a condition of a sample more reliably and/or correctly more often than known machine learning models, since the contrast of the OCT signal can be significantly enhanced at depth and reveal mesoscale features obscured by the limits of detection noise.

That the first training collection path is spatially and/or angularly offset from the illumination path by a first training offset can be used for optimizing contrast at a particular depth. With increasing spatial or angular offset, details at greater depths will emerge in the images.

However, with increasing spatial or angular offset, details closer to the surface of the sample may decrease in intensity or may disappear altogether. The two training images, one training image with an offset and one training image with a smaller offset or no offset will supplement each other so that details of the structure in the sample can be determined from the surface down to depths not possible to reach with a conventional OCT without offset.

In addition, it has turned out that when the offset is increased, larger structures or larger particles in the sample can be made visible, so that the two training images, one training image with an offset and one training image with a smaller offset or no offset will supplement each other to show more details-both structures of somewhat smaller sizes and structures of somewhat larger sizes.

When training the model, combinations of the first training image, the second training image, and the known condition are provided to the model, so that the trained model is able to provide a correct condition or a probability for a certain condition based on a first image and a second image of a sample with an unknown condition.

The known condition can be binary, where the known condition of the sample is that the sample has the condition or does not have the condition. The known condition can be a number and unity of a quantity, where the number can be within a certain range or a quality related to the quantity. The quantity can e.g. be elastic modulus, shear modulus, Young's modulus, or strength of e.g. a polymer sample. Micro-cracks in the polymer or defects in the structure already when casting/molding or due to subsequent stress may lower the strength of the material of the sample. However, the extension or amount of micro-cracks or defects as well as the relative position of the micro-cracks may be hard for the naked eye to determine because of turbidity. By providing the model with the known conditions associated with corresponding first training images and second training images, the model will be able to e.g. assess whether a specific sample possesses the required properties for its intended use.

The condition can e.g. also relate to a medical condition or medical diagnosis. The known condition can then be a certain disease or not that disease. The known condition can be how far the disease, for example an early stage tumor, has progressed, the known condition e.g. can be no indication of the disease, a very early or initial stage of the disease, a medium stage of the disease, an advanced stage of the disease that will need immediate treatment to the save the subject/patient, and so on. These levels can of course be further elaborate than presented here. The unknown condition to be determined can be a probability for the condition, like medical condition or diagnosis, or the probability for the subject not having the condition. The unknown condition to be determined can be a probability for a certain stage of the condition or probabilities for several certain stages of the condition. Based on that information the physician, if the condition is a medical condition or diagnosis, will be able to decide a final medical condition of the subject, maybe with the assistance of other information.

The image(s) can be an A-scan of the sample, where A-scan is the (1D) depth profile of the sample showing scattering at the surface of the sample and/or at a certain depth and/or up to a certain depth from the surface of the sample. The 2D image(s) can be a B-scan (a cross-sectional tomogram comprised by adjacent A-scans) of the sample, where a cross-sectional view along a stretch of the sample is shown, where scattering ability of the sample is shown in the cross-section.

That the first training collection path is angularly offset from the illumination path by a first training offset can mean that the illumination path is non-parallel to the first training collection path and/or the second training collection path.

The disclosure also relates in a second aspect to a method of determining an unknown condition, wherein the method comprises the steps of illuminating a sample along an illumination path, capturing a first image of the sample along a first collection path, wherein the first collection path is spatially offset and/or angularly offset from the illumination path by a first offset, and capturing a second image of the sample along a second collection path different from the first collection path, determining an unknown condition based on the first and second images using a machine learning model trained according to any aspect or embodiment of the first aspect as presented in the present application.

The unknown condition of the second aspect is of the same type as the known condition of the first aspect. That the unknown condition of the second aspect is of the same type as the known condition of the first aspect does not necessarily mean that the outcome of the unknown condition (e.g. positive or negative regarding a certain diagnosis or state) is the same as for the known condition, but only that the unknown condition and the known condition relate to the same type of condition.

The disclosure also relates in a third aspect to a method of determining segmentation in a sample, wherein the method comprises the steps of illuminating a sample along an illumination path, capturing a first image of the sample along a first collection path, wherein the first collection path is spatially offset and/or angularly offset from the illumination path by a first offset, capturing a second image of the sample along a second collection path different from the first collection path, and relating the first image and the second image to each other for determining segmentation in the sample.

As mentioned above regarding the first aspect of the disclosure, that the first collection path is spatially and/or angularly offset from the illumination path by a first offset can be used for optimizing contrast at a particular depth. With increasing spatial or angular offset, details at greater depths will emerge in the images. However, with increasing spatial or angular offset, details closer to the surface of the sample may decrease in intensity or may disappear altogether. The two images, one image with an offset and one image with a smaller offset or no offset will supplement each other so that details of the structure in the sample can be determined from the surface down to depths not possible to reach with a conventional OCT without offset.

In addition, it has turned out that when the offset is increased, larger structures or larger particles in the sample can be made visible, so that the two images, one image with an offset and one training image with a smaller offset or no offset will supplement each other to show more details-both structures of somewhat smaller sizes and structures of somewhat larger sizes.

Relating the first image and the second image to each other a more detailed information about the sample and at a deeper level can be achieved than in known methods.

With any of the first, second or third aspects, image formation at depths beyond several scattering mean free paths, typically beyond 1 or 2 mm that has been a major limitation of OCT, can be overcome.

Using SO-OCT, we demonstrate that we can achieve greater depth imaging while increasing the proportion of collected multiply scattered light. This is achieved by the preferential collection of multiply scattered light, counteracting conventional exponential attenuation of intensity with depth by the increasing likelihood of multiple scattering events needed to reach the offset detector aperture. SO-OCT also allows the reduction of the ballistic component from close to the sample surface, which enables further optimization of the dynamic range of the detector to the signal deeper in the sample which cannot be achieved using structured beams alone.

Furthermore, due to the spatial offset, a different profile of the scattering phase function is sampled using SO-OCT. The spatial offset may increase contrast depending on the various factors that impact the scattering properties. In particular, the spatial offset enhances the contrast of mesoscale features, i.e., features much larger than the wavelength, relative to bulk scattering from structures smaller than the wavelength.

Additionally, by imaging at a collection of offsets, the angular distribution of the scattering function may be recovered. This may enable a new form of contrast in OCT, or even the spatial discrimination of the mean scatterer cross-section in samples. Sequences of spatially offset B-scans could also be compounded to preserve the clarity at low depth and signal strength at depth.

Due to the simple geometry used in SO-OCT, this technique can be easily combined with other approaches, such as adaptive optics and beam shaping, in order for the illumination light to penetrate into turbid samples deeper. Even longer wavelengths than 800 nm and 1300 nm may also have improved imaging depth with SO-OCT. Wavelengths above 800 nm would be beneficial, preferably wavelengths above 1200 nm and even above 1500 nm or above 2000 nm would be beneficial.

Greater imaging depth and contrast were achieved despite increasing the contribution of multiple scattering.

The offset can be tuned to optimize contrast at a particular depth in the sample (FIG. 5c). The optimal offset parameters and achievable enhancement of contrast-to-noise ratio (CNR) may depend on the scattering properties of the sample. The modelling presented here also provides a platform for extracting the scattering properties directly from the OCT images. The enhancement of mesoscale structures in the image could also aid in identifying different structures and boundaries between types of tissue in scattering biological samples. So by capturing a first image and a second image having different spatial offsets in relation to the path of the illumination light, different depths can be characterized. If e.g. the spatial offset for one of the first image and the second image is zero or low like less than half the diameter of the spot illuminated on the sample by the illumination light, then structures or segmentation close to the surface of the sample will be determined. If e.g. the spatial offset for the other of the first image and the second image is a bigger like more than half the diameter of the spot illuminated on the sample by the illumination light,

Further, our offset geometry adds additional contrast from collecting a varied proportion of scattering angles from the sample.

The disclosure also relates in a fourth aspects to an optical coherence tomography system, wherein the system comprises a light source configured to illuminate a sample along an illumination path, at least one optical instrument for capturing a first image of the sample along a first collection path, wherein the first collection path is spatially and/or angularly offset from the illumination path by a first offset, and for capturing a second image of the sample along a second collection path different from the first collection path, and a processor configured to determine an unknown condition based on the first image and the second image using a machine learning model trained according to any aspect or embodiment of the first aspect as presented in the present application.

The optical instrument for the OCT can be an interferometer, like e.g. a Michelson interferometer or a Mach-Zehnder interferometer, with a spectrometer or a detector like a 1D array or a 2D array for detecting incoming radiation from the sample through the optical instrument.

The optical coherence tomography system will have all the advantages presented regarding the first, second and third aspects.

Regarding the first, second, third and fourth aspects of the disclosure, the pairs of terms
first training image and first image,
second training image and second image,
first training collection path and first collection path,
second training collection path and second collection path,
first training offset and first offset, and
second training offset and second offset, respectively
   may be understood interchangeably.

The embodiments presented below may relate to any or all of the four aspects of presented above unless presented otherwise.

In an embodiment, the first training image and the second training image may be captured by an image capturing device, such as an image sensor or a camera, like a CCD camera, or by a photodetector, like a photodiode. The imaging capturing device may be a so-called time-domain system or frequency-domain OCT system, including spectral-domain OCT using spectrometers and 1D/2D image sensors, or swept-source OCT in which the image is detected by means of a photodetector.

The image sensor, the camera, and/or the CCD camera may capture a two dimensional first training image and a two dimensional second training image

An image like the first training image and/or the second training image and/or any other training image may or may not be understood to comprise an output or a series of outputs from the photodiode positioned in the first training collection path and/or the second training collection path and/or any other training collection path. As mentioned above, the photodiode may be used for swept-source OCT.

In an embodiment, the method may comprise the steps of capturing a series of training images, wherein each of the training images of the series of training images has a training collection path different from the training collection paths of the other training images, wherein the series of training images comprises the first training image and the second training image, and providing the series of training images as input to the model. The series of training images may comprise three, four, five, six, seven, eight, nine, ten, or more training images.

The series of training images may be a discreet series of training images, wherein each of the training images may be captured in a shot or an exposure.

The fact that each of the training images of the series of training images has a training collection path different from the training collection paths of the other training images may mean that each training collection path has a spatially and/or angularly offset that is different from the offsets of the other training collection path.

The series of training images captured at different offsets will provide information about the first sample at different depths, wherein a higher offset may provide information at a deeper depth, so that the series of training images will feed the machine learning model information like structure from the surface of the first sample to far deep in the first sample. If the relevant information in the first sample connected to the known condition is a combination of structure at the upper part of the first sample and at a deeper level of the first sample, the series of training images fed to the machine learning model will be a necessity to train the machine learning model. If it is important to know at which depth the information, like structure, can be found, then the series of training images-which comprises information about, where the surface level of the first sample is, and comprises information about the first sample at different levels from the surface level-will provide the necessary information for the machine learning model.

In an embodiment, the series of training images may be captured in one single exposure. A photosensitive diode of a photodiode, or a one or two-dimensional photosensitive chip of a CCD camera connected to a spectrometer may provide a continuous output signal or continuous output signals, which is/are varying in response to variations in intensity of a radiation falling on the photosensitive diode or on the two-dimensional photosensitive chip. The single exposure may provide a B-scan of the first sample, which will provide a fastest possible imaging of the cross-section of the first sample.

The merged images can be formed by capturing the images in a single exposure or by combining several images in post-processing and using the resulting merged image as the training image.

The one single exposure may include all of the offsets multiplexed in time for a single A-scan that together will build up a B-scan.

Alternatively, the one single exposure may build up a composite A-scan with all the offsets combined by tuning the dwell time per offset to achieve best contrast over a large depth range.

In one embodiment, the method may comprise the step of moving the image capturing device in relation to the first sample from the first training collection path to the second training collection path for capturing the series of training images. Moving the image capturing device in relation to the first sample may also be achieved by moving the first sample in relation to the image capturing device. The first training collection path and/or the second training collection path may or may not have a training offset relative to the illumination path.

In an embodiment, the method comprises the steps of capturing a third training image of the sample along a third training collection path different from the first and second training collection paths for providing a second reference image, optionally capturing a fourth training image of the sample along a fourth training collection path different from the first, second, and third training collection paths for providing a third reference image, and training a machine learning model based on the first, second, and third training images, optionally also based on the fourth training image, and the known condition.

In an embodiment of any of the first and fourth aspects, the machine learning model can be a deep learning model or a neural network.

In an embodiment of any of the first and fourth aspects, the condition can be the ground truth when training the model.

In an embodiment of any of the first, second, third and fourth aspects, the sample can be a tissue of a subject. The condition may then relate to the subject and/or the tissue and be a medical condition or diagnosis.

In an embodiment of any of the first, second, third and fourth aspects, at least a first point in the first training image can be correlated to at least a second point in the second training image. With a larger spatial and/or angular offset, the images/training images will show deeper layers of the sample as well as larger structures and larger particles. By choosing the first point in the first training image and the second point in the second training image, where the first point and the second point correspond to a same point in the sample, first information gained from the first training image and second information from the second training image can be more easily correlated to each other so that the first information and the second information can be combined in a more effective way.

In an embodiment of any of the first, second, third and fourth aspects, at least two first points in the first training image can be correlated to at least two second points in the second training image. Compared to relating the first point and the second point to each other as presented above, relating to two first points to two second points to each other, the two first points and the two second points will first information gained from the first training image and second information from the second training image can be more easily correlated to each other so that the first information and the second information can be combined in a more effective way, since one of the two first points in the first training image related to one of the two second points in the second training image, and the other of the two first points in the first training image related to the other of the two second points in the second training image, there is no rotational degree of freedom between the first training image and the second training image or between the first training image and the second training image.

In an embodiment of any of the first, second, third and fourth aspects, the second training collection path can be along the illumination path. When the second training collection path or the second collection path is along the illumination path, the captured second training image or second image will correspond to an image captured by a conventional OCT without an offset. Such a second training image or second image will be a suitable supplement to the first training image or first image, respectively, with the first training offset or first offset, since then the structures of the sample can be characterized from the surface and deeper into the sample than compared to by a conventional OCT.

In an embodiment of any of the first, second, third and fourth aspects, the second training collection path can be spatially offset and/or angularly offset from the illumination path by a second training offset different from the first training offset.

In an embodiment of any of the first, second, third and fourth aspects, the first training offset or the first offset can be more than 5 μm, preferably more than 10 μm, most preferably more than 20 μm.

In an embodiment of any of the first, second, third and fourth aspects, the first training offset or the first offset can be less than 250 μm, preferably less than 200 μm, most preferably less than 150 μm.

In an embodiment of any of the first, second, third and fourth aspects, the sample can be illuminated by a light ray having a spot diameter on the sample, wherein the first training offset or the first offset can be at least half the spot diameter or at least the spot diameter, preferably more than two times the spot diameter, more preferably more than three times the spot diameter, even more preferably more than four times the spot diameter, and most preferably more than five times the spot diameter.

The spot diameter can be a focal spot diameter if the light ray is focused down on the sample.

In an embodiment of any of the first, second, third and fourth aspects, the sample can be illuminated by a light ray having a spot diameter on the sample, wherein the first training offset or the first offset can be less than 50 times the spot diameter, preferably less than 30 times the spot diameter, more preferably less than ten times the spot diameter, and most preferably less than eight times the spot diameter.

In an embodiment of any of the first, second, third and fourth aspects, the method can comprise steps of capturing a plurality of images of the sample along a plurality of different training collection paths, wherein the plurality of different training collection paths are spatially offset and/or angularly offset from the illumination path by a plurality of different training offsets, and providing the plurality of images as input to the deep learning model.

In an embodiment of any of the first, second, third and fourth aspects, the illumination path and the first training collection path can form an angle between 5° and 90°, preferably between 10° and 70°, more preferably between 15° and 50°, and most preferably between 20° and 40°.

In an embodiment of any of the first, second, third and fourth aspects, the illumination path and the second training collection path can form an angle between 5° and 90°, preferably between 10° and 70°, more preferably between 15° and 50°, and most preferably between 20° and 40°.

In an embodiment of the fourth aspect, the model can be configured to provide a condition based on a first image of the sample captured along a first collection path, wherein the first collection path is spatially offset and/or angularly offset from the illumination path by a first offset, and a second image of the sample along a second collection path.

In an embodiment of the fourth aspect, the system can be configured to control the optical system to change the spatial and/or angular offset of the collection path.

In an embodiment, the optical coherence tomography image (cross-sectional tomogram) may be achieved by an interferometer with two optical paths, wherein the first training image comprises a number of first training images for different optical path length differences between the two optical paths.

The number of first training images for different optical path length differences will provide information at different depths of the first sample.

In an embodiment, the first training image for training the machine learning model may be a first cross-sectional tomogram based on the number of first training images.

In an embodiment, the second training image may comprise a number of second training images for different optical path length differences between the two optical paths.

The number of second training images for different optical path length differences will provide information at different depths of the first sample.

In an embodiment, the second training image for training the machine learning model may be a second cross-sectional tomogram based on the number of second training images.

In an embodiment, the second training image may comprise a number of second training images for different optical path length differences between the two optical paths, wherein a fifth training image may comprise a number of fifth training images for different optical path length differences between the two optical paths, wherein the second training image for training the machine learning model may be a compound of a second cross-sectional tomogram based on the number of second training images and a fifth cross-sectional tomogram based on the number of fifth training images.

The fifth training image and the fifth training images does not necessarily require that there are third and fourth training image(s). The term “fifth” is only used to distinguish the term from the third and fourth training image(s) mentioned earlier.

The method according to any of the preceding claims, wherein the step of illuminating the first sample may be performed by radiation having a narrow, instantaneous line width of less than 0.1 nm, preferably less than 0.05 nm, more preferably less than 0.01 nm, most preferably less than 0.001 nm.

In an embodiment, the radiation irradiating the first sample may have a narrow, instantaneous line width that is tunable, so that the first sample is irradiated by different wavelength ranges for the first training image and the second training image (or for each of the series of training images), or so that the first sample is irradiated by different wavelength ranges for several first training images and/or so that the first sample is illuminated by different wavelength ranges for several second training images. The scattering, absorption, reflection and/or transmission by the first sample may differ depending on the wavelength range, so that the tunable narrow bandwidth may provide different information about the first sample depending on around which wavelength tunable narrow bandwidth is centered.

In an embodiment, the narrow line width may be swept from a first wavelength to a second wavelength different from the first wavelength.

A tunable laser can provide the narrow line width that is swept from the first wavelength to the second wavelength.

In an embodiment, the illumination path may be swept over the first sample from a first position to a second position different from the first position.

The first training image may be captured by a photodiode and may comprise the positions of the sample scanned between the first position and the second position.

In an embodiment, the first training offset and/or the second training offset may be constant, or at least partly constant, during the scanning of the illumination path.

In an embodiment, the first training offset may be varied/moved spatially and/or angularly during the step of capturing the first training image of the sample along the first training collection path from a first training offset position to a second training offset position differently from the first training offset position.

In an embodiment, the second training collection path may be swept from a first training collection path position to a second training collection path position differently from the first training collection path position.

### DESCRIPTION OF THE DRAWINGS

FIG. 1a is a schematic view of a first spatially offset optical coherence tomography (SO-OCT) system;

FIG. 1b is a schematic view of a second spatially offset optical coherence tomography (SO-OCT) system;

FIG. 2a is a schematic view of a sample where the collected light is spatially offset from the illumination light;

FIG. 2b is a schematic view of a sample where the collected light and the illumination light are angularly different and where the collected light and the illumination light are spatially offset at the upper part of the volume of interest but not at the lower part of the volume of interest;

FIG. 2c is a schematic view of a sample where the collected light and the illumination light are angularly different and where the collected light and the illumination light are spatially offset in the volume of interest;

FIG. 3a shows graphs showing the dependence of the OCT intensity at different depth for different spatial offsets;

FIGS. 3b-3e show B-scans for spatial offsets of 0 μm (FIG. 3b), 25 μm (FIG. 3c), 50 μm (FIG. 3d), and 75 μm (FIG. 3e);

FIGS. 4a-4d show Ti0₂ microparticles with mean diameter <5 μm embedded in a PDMS gel, acquired with the second SO-OCT system presented in FIG. 1b, for offsets at s=0 μm (FIG. 4a), 30 μm (FIG. 4b), 40 μm (FIG. 4c), 50 μm (FIG. 4d);

FIGS. 5a and 5b show B-scan images with (a) no offset and (b) with a spatial offset of s=40 μm;

FIG. 5c is a graph showing the OCT signal intensity averaged across the lateral direction vs depth and normalized to the surface intensity at different offsets; and

FIG. 6 shows calculations of the heterodyne efficiency factor (Y) in conventional OCT and SO-OCT.

### A MORE DETAILED DESCRIPTION OF SOME OF THE DRAWINGS

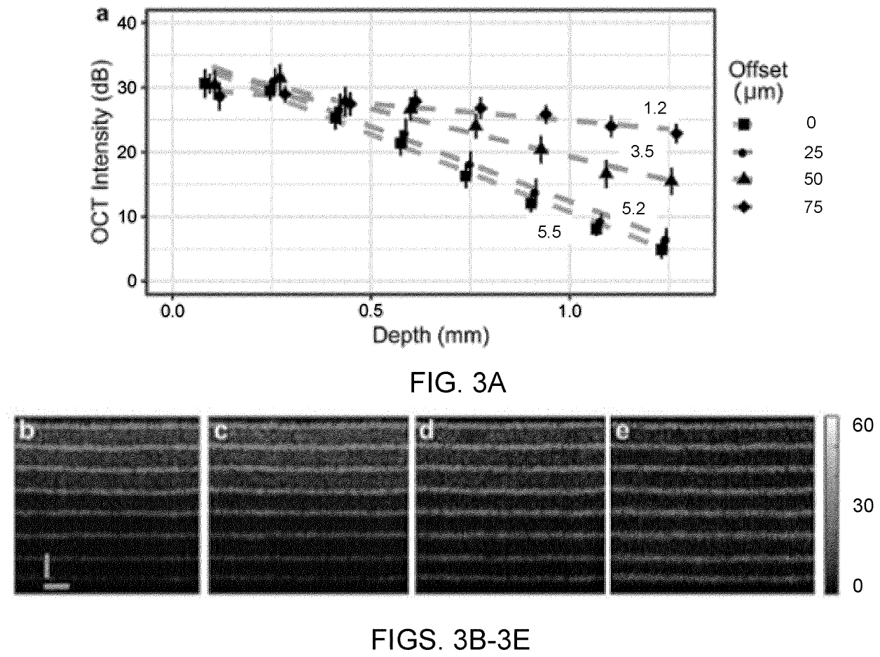

FIG. 3 SO-OCT demonstrates reduced attenuation with increasing offset in nescofilm phantoms. (a) Averaged OCT intensity across A-scans at the layer-layer interface as a function of optical depth in a 7-layer nescofilm phantom imaged at different spatial offsets. Data points with different offsets are slighted shifted in the x-coordinate for clarity. Error bars correspond to the standard deviation of OCT intensity. Dashed lines represent a linear fit to the log-intensity, and the corresponding number is a rough approximation of the effective attenuation coefficient (mm-1). (b-e) show the corresponding B-scans for offsets of s=0, 25, 50, 75 μm, respectively. Scale bars indicate 200 μm, and the intensity scale in (b-e), is OCT intensity in dB. Images were acquired with a central wavelength of 800 nm.

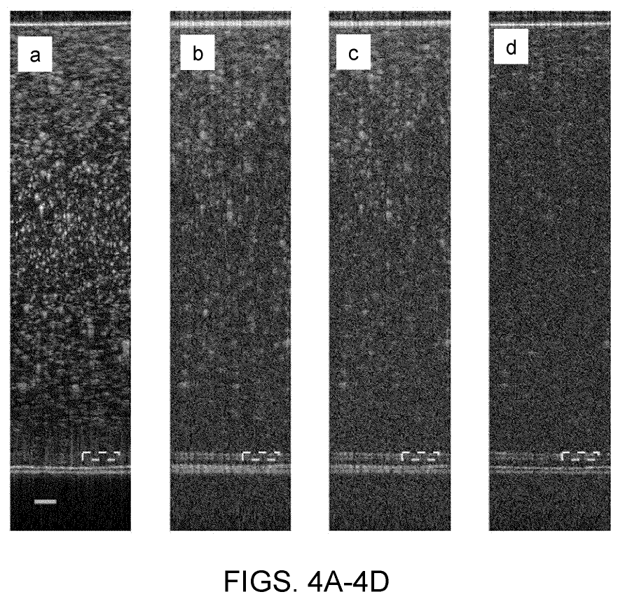

FIG. 4A phantom experiment demonstrates that SO-OCT provides contrast based on scattering properties. FIGS. 4a-d Ti0₂ microparticles with mean diameter <5 μm embedded in a PDMS gel, acquired with the second SO-OCT system, for offsets at s=0, 30, 40, 50 μm, respectively. Yellow boxes (lower) and red boxes (higher) indicate the regions used to calculate the CNR. Scale bars indicate 200 μm.

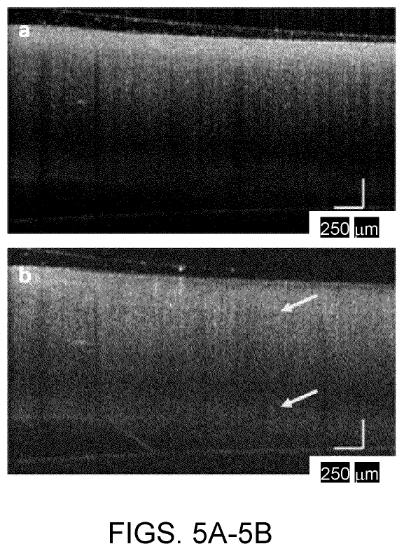

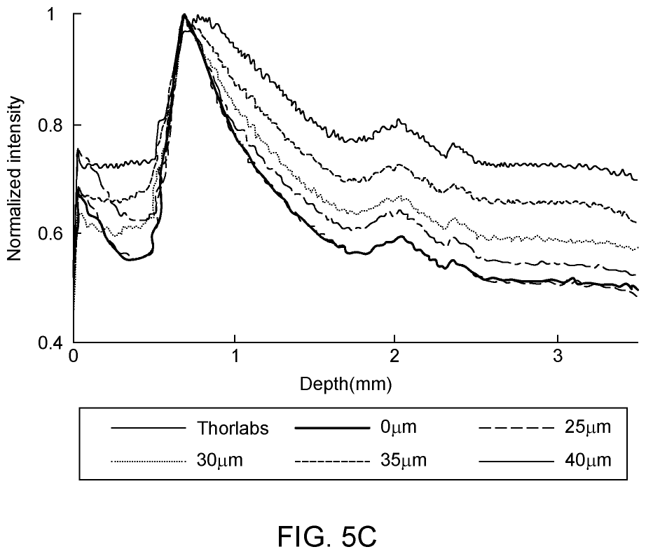

FIGS. 5a-c OCT B-scan images of an ex vivo mouse femur with (a) conventional OCT and (b) s=40 μm acquired with the second SO-OCT system centred at 1295 nm. The SO-OCT demonstrates enhanced contrast between tissue layers. The yellow arrows indicate the boundary between the hard bone and marrow, and the red arrow indicates the bottom surface of the bone, which is revealed by the enhanced contrast in the SO-OCT image. (c) shows the OCT signal intensity averaged across the lateral direction vs depth and normalized to the surface intensity at different offsets. The line labelled ‘Thorlabs’ indicates the signal collected through the standard setup without the additional components for SO-OCT. We can clearly observe a reduction in the total effective attenuation as the offset is increased, which leads to improved CNR at depth.

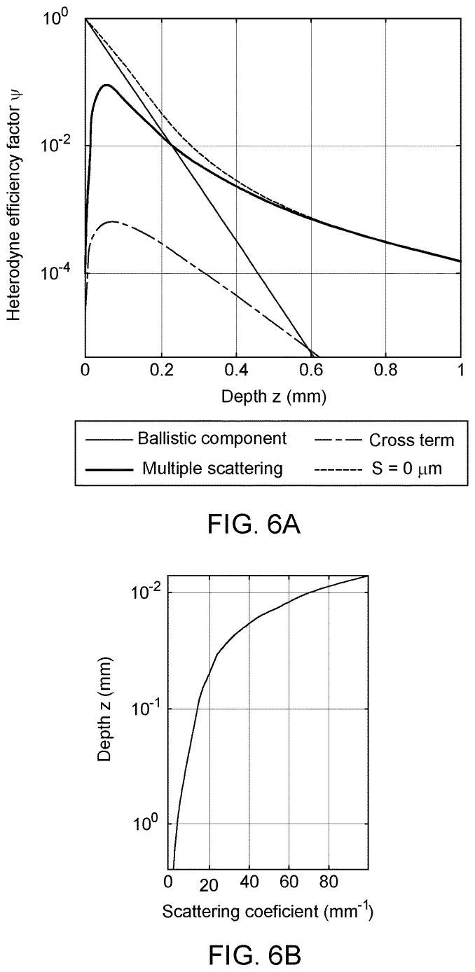

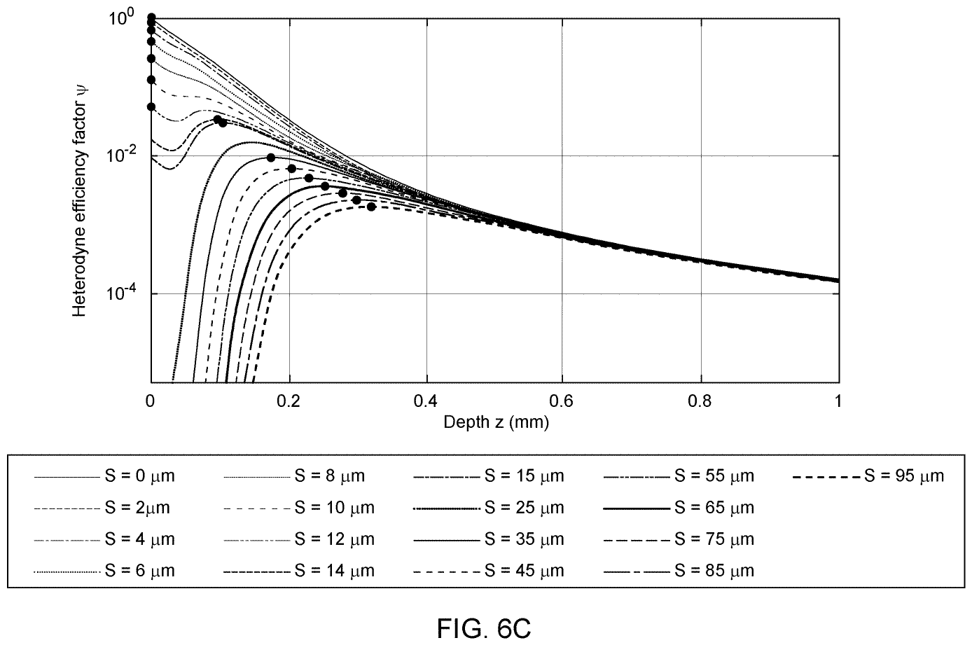

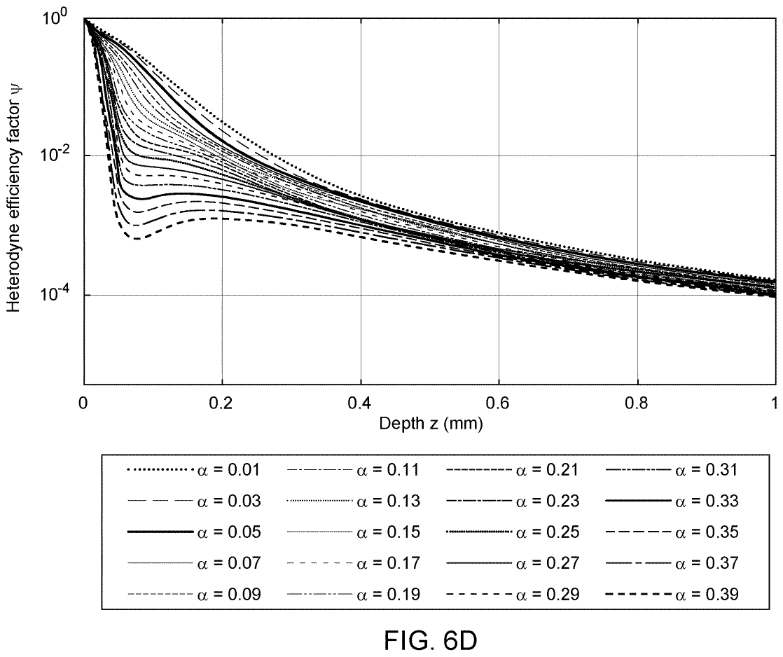

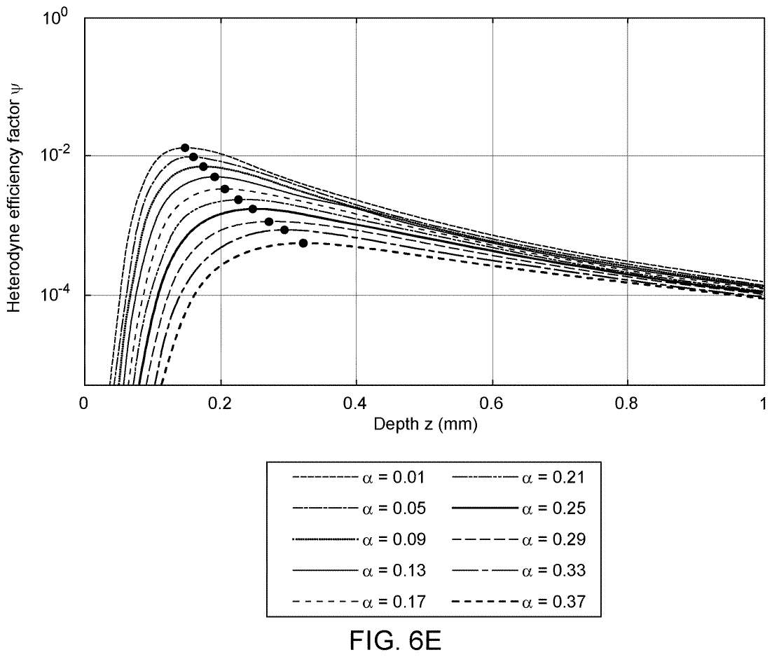

FIG. 6 Calculations of the heterodyne efficiency factor (ψ) in conventional and SO-OCT. (a) shows the relative contribution of ballistically scattered and multiply scattered light in image formation in conventional OCT. While the ballistic signal dominates superficially, the multiply scattered component rapidly dominates the signal at depth. (b) shows the depth at which the multiply scattered light dominates the OCT signal for various scattering coefficients. (c) is the heterodyne efficiency factor in SO-OCT with α=0 and various offsets. The circles plotted on each line indicate the depth with maximum contrast. (d) is the heterodyne efficiency factor with s_0=0 and various angular offsets. The blue dot in the upper left corner indicated the depth with highest contrast for all angular offsets. (e) shows the heterodyne efficiency factor incorporating both the angular and lateral offset with s_0=25. Note that the darkest green line in (e) should almost match the line for s=25 μm in (c). The circles plotted on each line indicate the depth with highest contrast. The following system parameters representing the system described in FIG. 5b were used for these calculations unless otherwise specified: λ_0=1295 nm; [symbol] ω [symbol] _0=1.5 mm; f=36 mm; n=1.4; μ_s=10 [symbol] mm [symbol] \hat{ }(−1); μ_a=0 [symbol] mm [symbol] \hat{ }(−1); θ_rms=0.3. All angles are in units of radians.

### DETAILED DESCRIPTION OF THE DISCLOSURE

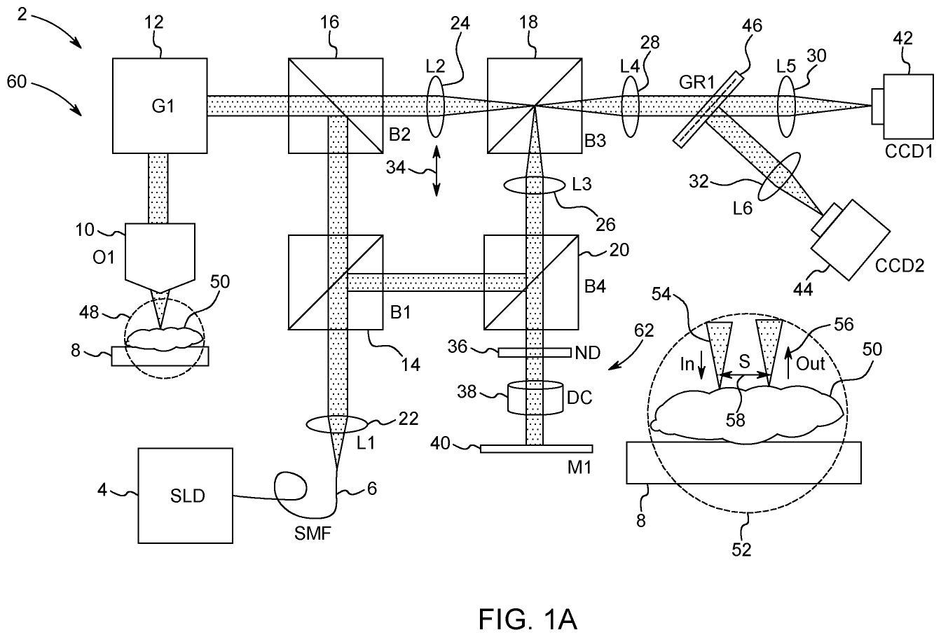

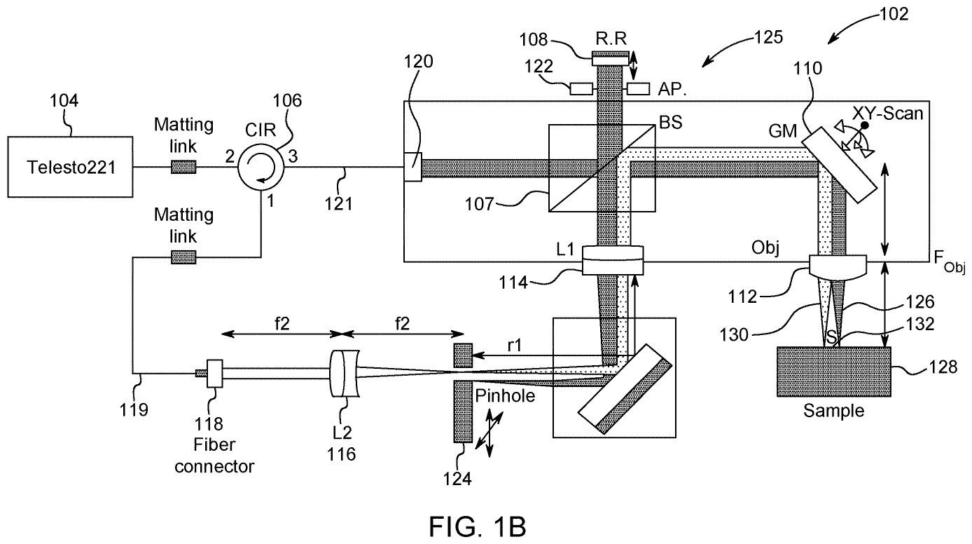

FIG. 1a shows a first spatially offset optical coherence tomography (SO-OCT) system 2. The first SO-OCT system comprises a superluminescent diode (SLD) laser (S850, Superlum, Cork, Ireland) with a central wavelength of 800 nm and a bandwidth of 14 nm as a light source 4. The first SO-OCT system 2 comprises a single mode fibre 6; a 3D stage 8; a first microscope objective 10 or a convex lens; a 2D galvo mirror 12 (GVSM002/M, Thorlabs); a first non-polarizing beamsplitter cube 14 (BS029, Thorlabs) with 90% reflection and 10% transmission; a second 16, a third 18, a fourth 20 non-polarizing beamsplitter cubes (BS014, Thorlabs) each with 50% reflection and 50% transmission; a first lens 22, a second lens 24, a third lens 26, a fourth lens 28, a fifth lens 30, and a sixth lens 32. The double arrow 34 indicates the direction of translation of the second lens 24. The first SO-OCT system 2 further comprises a neutral density filter 36; a dispersion compensation 38 (LSM03DC, Thorlabs); a reference mirror 40, a first CCD camera 42 (FL3-U3-32S2M-CS, Point Grey), and a line CCD camera 44 (Aviiva EM1, Teledyne); a grating or a transmission grating 46 (1200 lines/mm, coated for 700 nm to 960 nm). A first dashed circle 48 shows a sample 50 positioned on the 3D stage 8. A second dashed circle 52 shows a magnification of the sample 50 positioned on the 3D stage 8. The second dashed circle 52 also shows illuminating light 54 directed from the light source 4 and collected light 56 collected from the sample 50 and directed to the first CCD camera 42 and the line CCD camera 44. The collected light 56 is collected at a first offset or first spatial offset, s, 58 from the path of the illuminating light 54.

The first offset 58 of the collected light 56 is adjustable by moving the second lens 24 in the direction of the double arrow 34, e.g. by positioning the second lens on a translation stage (not shown). In other words, the first offset 58 of the collected light 56 that is collimated by the first objective 10 and further guided to the first 42 and the line 44 CCD cameras will depend on the vertical position of the second lens 24. The linear relationship between the shift of the second lens 24 and the first spatial offset 58 can be calibrated carefully in advance by measuring the laser spot on the first CCD camera 42.

Since the first SO-OCT system comprises a sample arm 60 and a reference arm 62, which are completely decoupled, the first spatial offset 58 can be freely tuned without influencing other optical paths.

The first objective 10 is a low NA microscope objective that is used for focusing the light up to e.g. 1 mm into the sample. The focus plane is imaged on the first CCD camera 42, which is positioned in the direction of the zeroth diffraction order of the grating 46. The first diffraction order is focused onto the line CCD camera 44, which provides the spectral information. The reference arm 62 propagates through the same optical path except for the dispersion compensation 38 before the light of the reference arm interferes with the light of the sample arm 60.

The first CCD camera 42 may be a first photodiode 42 and/or the line CCD camera 44 may be a second photodiode 44 so that the optical coherence tomography (SO-OCT) system 2 can be used for swept-source OCT.

FIG. 1b shows a second spatially offset optical coherence tomography (SO-OCT) system 102. The second SO-OCT system 102 comprises a unit 104 comprising a superluminescent diode (SLD) laser (not shown) as a light source emitting at 1295 nm and a spectrometer (not shown) as a detector, where the spectrometer can e.g. be a linear InGaAs array-based spectrometer or another spectrometer commonly used for OCT systems.

The second SO-OCT system 102 further comprises a circulator 106; a fifth beamsplitter 107; retroreflector 108; galvo mirror 110; a second microscope objective 112 or a convex lens; a seventh lens 114, an eighth lens 116 for collimating light, a ninth lens 118 for focusing the light from the eighth lens 116 into a first light fibre 119, a tenth lens 120 for collimating light coming out of a second light fibre 121; a variable aperture 122 for adjusting the power level of a reference beam in the reference arm; a movable pinhole 124. The fifth beamsplitter 107 and the retroreflector 108 together form a Michelson interferometer 125.

Illuminating light 126 from the light source is focused by the second microscope objective 112 on a sample 128. Collected light 130 from the sample is directed to the spectrometer. There is a spatial offset, s, 132 between the path of the illuminating light 126 and the path of the collected light 130. The spatial offset of the collected light detected by the spectrometer will depend on the position of the pinhole 124. The pinhole 124 is preferably positioned on a 1D or 2D translation stage (not shown) for by translating the pinhole tune the offset 132 of the collected light 130.

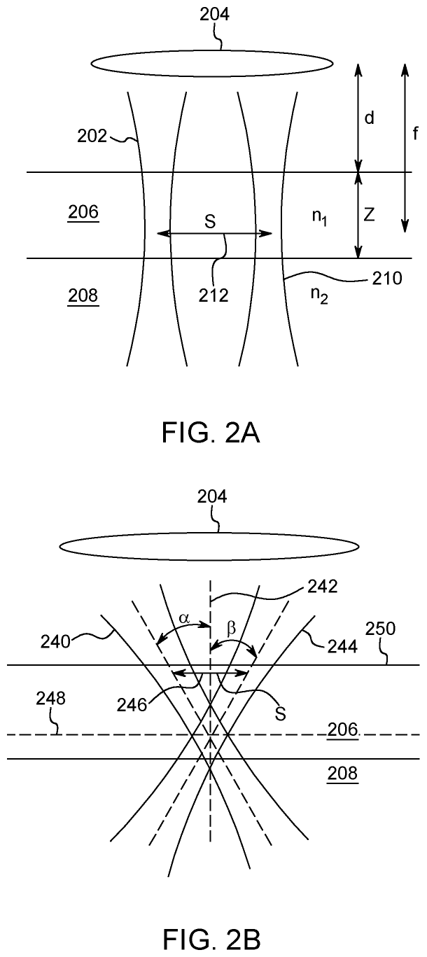

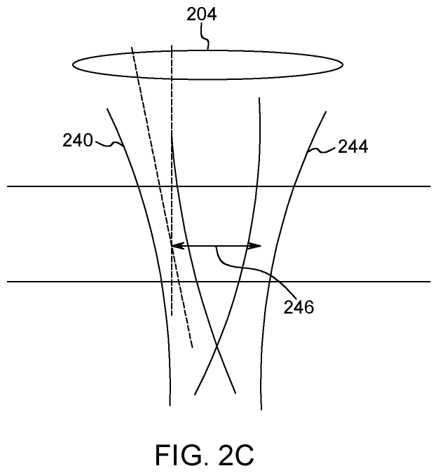

FIG. 2a shows illuminating light 202 focused by a lens 204 on a first tissue layer 206 with a first refraction index n₁ above a second tissue layer 208 with a second refraction index n₂. The lens 204 has a focal length, f, and is positioned a distance, d, above the first tissue layer 206. Collected light 210 from the tissue with a spatial offset, s, 212 from the illuminating light 202 is collected by the lens 204 and further transferred to a detector (not shown). As shown above, the spatial offset 212 of the collected light 210 will depend on e.g. the position of the second lens 24 or the position of the pinhole 124 or a third alternative depending on the set-up.

The lens 204 should also be understood to be a schematic representation and the illuminating light and the collected light may e.g. pass through different lenses or objectives.

FIG. 2b shows the lens 204 above the first tissue layer 206 on top of the second tissue layer 208 like in FIG. 2a. However, illuminating light 240 forms a first angle, α, with a normal 242 of the first tissue layer 206 and collected light 244 forms a second angle, β, with the normal 242, where the first angle and the second angle may or may not be the same angle.

The size of the first angle, α, will depend on how the illuminating light is directed by the optics before passing through the lens 204. The direction of the light prior to the lens 204 will form an angle with the normal 242. The size of the first angle, α, will depend on how the illuminating light is directed by the optics before passing through the lens 204. The direction of the light prior to the lens 204 will form an angle with the normal 242.

The size of the second angle, β, will depend on how the optics is arranged after the lens 204 for collecting the collected light. The direction of the light after the lens 204 towards the optics collecting the collected light will form an angle with the normal 242. The size of the second angle, β, may be tuned by moving the optics, so that the desired direction of the collected light is directed to the detector.

Since the illuminating light 240 forms the first angle, α, with the normal 242 and the collected light 244 forms the second angle, β, with the normal 242, the offset, s, 246 of the collected light 244 in relation to the illuminating light 240 will depend on the depth position in the tissue. At a first depth level 248, which in this case corresponds to the focal plane of the lens 204, the offset is zero. If the second angle is equal to the first angle, the offset can be described by

s(z)=z tan α+s₀
where s₀ is the offset at a top layer 250 of the first tissue layer and z is the distance from the top layer 250, where z has a negative sign in the down direction. When z corresponds to the first depth level 248, then z tan α is equal to −s₀, and the offset is zero.

FIG. 2c corresponds to the situation presented in FIG. 2b, with the difference that at the focal plane of the lens 204 the offset is not zero.

In an embodiment of the first aspect, the disclosure relates to a method of using optical coherence tomography for training a machine learning model, the method comprising the steps of illuminating a first sample having a known condition along an illumination path, capturing a first training image of the sample along a first training collection path, wherein the first training collection path is spatially and/or angularly offset from the illumination path by a first training offset, optionally capturing a second training image of the sample along a second training collection path different from the first training collection path for providing a reference image, and training a machine learning model based on the first training image, the known condition, and optionally on the second training image. This embodiment of the first aspect can be combined with any other embodiment presented in the present application.

In an embodiment of the second aspect, the disclosure also relates to a method of determining an unknown condition, wherein the method comprises the steps of illuminating a sample along an illumination path, capturing a first image of the sample along a first collection path, wherein the first collection path is spatially and/or angularly offset from the illumination path by a first offset, and optionally capturing a second image of the sample along a second collection path different from the first collection path, determining an unknown condition based on the first image, and optionally the second image, using a machine learning model trained according to any aspect or embodiment of the first aspect as presented in the present application. This embodiment of the second aspect can be combined with any other embodiment presented in the present application.

In an embodiment of the fourth aspect, the disclosure also relates to an optical coherence tomography system, wherein the system comprises a light source for illuminating a sample along an illumination path, at least one optical instrument for capturing a first image of the sample along a first collection path, wherein the first collection path is spatially and/or angularly offset from the illumination path by a first offset, and optionally for capturing a second image of the sample along a second collection path different from the first collection path, and a processor configured to determine an unknown condition based on the first image, and optionally on the second image using a machine learning model trained according to any aspect or embodiment of the first aspect as presented in the present application. This embodiment of the fourth aspect can be combined with any other embodiment presented in the present application.

### EXAMPLES

### Example 1

We have imaged a phantom comprising seven layers of nescofilm (120 μm in thickness for one single layer) using the first SO-OCT system shown in FIG. 1a. By translating the lens L2, B-scans were acquired over a range of ~1 mm with varying offsets s=0, 25, 50, 75 μm. The exposure time of the camera (CCD) was tuned to achieve the same intensity of the reconstructed OCT signal at the first interface for fair comparison. FIG. 3a shows the mean and standard deviation with respect to the lateral direction of the averaged OCT intensity at each interface imaged at varying offsets. The corresponding B-scans are visualized in FIGS. 3b-e. We can clearly observe a reduction in the total effective attenuation as the offset is increased, which leads to an evident improvement in the layer-layer interface contrast with depth.

In low scattering media or at shallow depths (i.e., within a few mean free path lengths), the well-known Beer-Lambert exponential function is a reasonable approximation of the attenuation of the incident beam, which is reflected by the linear decay of the log-intensity when the spatial offset s=0. We can estimate the mean free path lₛ of nescofilm as 180 μm based on the intensity attenuation in standard OCT images. However, the probability of the number of scattering events comprising the local OCT signal increases with depth, and so does the heterodyne detection efficiency of the multiply scattered light (FIG. 6a). As a consequence, in SO-OCT, attenuation is counteracted because the offset ensures a preferential collection of light that has undergone a few scattering events. From the linear gradient of intensities, the effective OCT attenuation coefficient can be approximated as 5.5, 5.2, 3.5 and 1.2 mm−1 for s=0, 25, 50, 75 μm, respectively. From FIG. 3, we see around a 4.6-fold decrease in attenuation at an offset, s=75 μm.

The effects of multiple scattering, relevant to turbid media i.e., most biological tissues of interest, in OCT have been investigated in great detail. For mainly forward scattering samples, the temporally dispersive effect of multiple scattering may be negligible over few millimetres. We can observe this via the broadening in the thickness of the nescofilm layer interface. At 1-mm depth, the broadening of the layer interface thickness was 2.9, 23.5, 47.0% for 25, 50 and 75 μm offsets, respectively, which represents a trade-off between attenuation and resolution.

### Enhanced Contrast in Microbead Phantoms

The offset in collection makes SO-OCT more sensitive to multiply scattered light and the angle-dependent scattering properties of the sample. We can consider each detection event in SO-OCT to comprise a few forward-scattering events that introduce temporal dispersion on the order of less than the coherence time of OCT. The probability of back-scattering at a given angle is determined by the scattering phase function. For sample scatterers of diameter d<<λ, this scattering can be described by Rayleigh scattering, which has a substantial chance to scatter in all directions; scatterers of d<λ, can be described by Mie scattering, with predominantly forward and back scattering as d→λ; and, scatterers d>λ are described by geometrical optics, which back-scatter with a broad range of angles²³. With increasing offset, the likelihood of collecting the back-scattered signal is shifted from the purely normal to off-axis back-scattering. Thus, objects that have similar normal back-scattering coefficients may have vastly different angle scattering properties, which are linked to their scattering cross-section.

As a consequence, the contrast between structures with different scattering properties is rendered differently in SO-OCT relative to conventional OCT; structures within the range of preferential off-axis scattering, hereby termed mesoscale structures, are rendered with better contrast. We demonstrate this principle with two experiments shown in FIG. 4.

### Example 2

Using the second SO-OCT system with central wavelength of 1295 nm described in FIG. 1b we employed a similar phantom comprised of 0.1% w/w Ti0₂ microparticles with mean diameter <5 μm uniformly dispersed in a PDMS gel. A ~1 mm slab of this phantom was placed on top of a flat piece of transparent epoxy. By translating the pinhole in the collection path, B-scans were acquired over a range of ~1 mm with varying offsets s=0, 30, 40, 50 μm. Since this system is based on a modification of a commercial system and operated through software provided by the manufacturer, the exposure time could not be freely adjusted, and we were unable to achieve the same sensitivity at the surface in all images. Instead, the images are normalized to the surface intensity. This limits the range of accessible offsets due to the simultaneous enhancement of the signal and noise floor during normalization. We note that the optimal offset for a particular sample will depend on the scattering properties; for some samples we may not have been able to achieve the optimal offset with this system. Despite this, it is clear from FIGS. 4a-4d that the bulk scattering contrast of the Ti0₂ microparticles, which fall within the predominantly forward and backscattering regime, is greatly reduced as the offset is increased, while the contrast of the bottom layer of the phantom relative to the bulk scattering is increased. To quantify this improvement, we calculated the contrast-to-noise ratio (CNR) for the regions indicated by the yellow box (signal) and the red box (background “noise”) for each B-scan. Here CNR is defined as

$$\vert μ - {μ}_{b} \vert / \sqrt{{δ}^{2} + {δ}_{b}^{2}} ,$$

where μ, δ, μ_{b}, and δ_{b} denote the mean and standard deviation of the pixel intensities in the signal region and background region, respectively. We measured values of 2.19 dB, 2.33 dB, 2.35 dB, and 2.40 dB for offsets of s=0, 30, 40, 50 μm, respectively, which demonstrates that the CNR at depth increases as the offset increases.

These two experiments, taken together, demonstrate the power of SO-OCT to reveal or enhance the contrast of hidden mesoscale structures within scattering samples. In fact, it is likely that with an off-axis geometry, substantial contrast enhancement can be achieved in samples that possess clearly separate scattering angle profiles, such as sub-resolution scattering media vs. beads, specular reflectors, or USAF targets.

### OCT Imaging in Mouse Bones

One of the main potential advantages of SO-OCT is the opportunity to improve CNR and imaging depth in highly scattering media for example, hard tissue like bone and cartilage. Only a few studies have investigated OCT in bone since the high multiple scattering severely limits the imaging depth. For this study we reverted to the second OCT system shown in FIG. 1b to image mouse bones to assess the opportunities for improving imaging in highly scattering tissue. Images of the bones with conventional OCT and s=40 μm offset are shown in FIGS. 5a and b, respectively. Visually, it is evident that the attenuation of the signal in the SO-OCT image is significantly less than in the conventional OCT image. Indeed, the attenuation between the top and bottom surface of the bone was reduced by over 24 dB for an offset of s=40 μm. The boundary between the hard bone and marrow are much more clearly defined, and the bottom surface of the bone is revealed. Additionally, we have plotted the OCT signal intensity averaged over the lateral direction and normalized to the surface intensity in FIG. 5c.

We note that we were able to achieve a similar contrast through the unmodified Thorlabs system and through the add on with no offset (s=0) although some signal was lost due to the additional optical components. The signal for the offset s=25 μm overlaps with the signal for s=0 μm because s=25 μm corresponds to an offset of less than the pinhole diameter, so some ballistically scattered light is still detected. In general, the contrast between the bottom and top surface of the bone is improved as the offset is increased. This is, in part, due to the ability to adjust the dynamic range of the image since the ballistic signal from near the sample surface is reduced. A slight broadening of the peaks is consistent with the trade-off between attenuation and resolution demonstrated in FIG. 3.

### Theoretical Example

Theoretical modelling of the physical principle of SO-OCT was performed using the extended Huygens-Fresnel (EHF) model. The EHF model is a wave-based model of image formation in OCT and an analytical solution to the scalar wave equation which, by its nature, can more accurately model the wave-like behaviour of light in tissue including interference and diffraction effects when compared with numerical methods like Monte Carlo simulations. It is the first OCT model that adequately includes both the ballistic and multiply scattered components of the signal. Additionally, the EHF model can more accurately model the impact of tissue optical properties on OCT image formation⁴⁰. This is particularly important for SO-OCT since the ideal imaging parameters may depend on the scattering properties of the sample.

There is ample experimental evidence that the EHF model yields a valid description of OCT image formation in scattering media in both the single and multiple scattering regimes. As such, a physical model of image formation in SO-OCT can be derived from an extension of the EHF model of a co-axial OCT system. We have already demonstrated similar extensions to the EHF model to consider the effects of multilayer scattering and to incorporate the absorption coefficient. Building on this work, we can derive expressions for the heterodyne efficiency factor and the signal-to-noise ratio as a function of depth for SO-OCT. The expression for the heterodyne efficiency factor with a separation s introduced between the illumination and collection paths, which, for now, are assumed to be parallel:

$$Ψ ( z , s ) = {e}^{- 2 {μ}_{a} z} [ {e}^{- 2 {μ}_{s} z} {e}^{\frac{- {s}^{2}}{2 {ω}_{H}^{2}}} + \frac{4 {e}^{- {μ}_{s} {z}_{( 1 - {e}^{- {μ}_{s} z} )}}}{( 1 + {μ}_{a} Δ {z}_{D} ) ( 1 + \frac{{ω}_{SA}^{2}}{{ω}_{H}^{2}} )} {e}^{\frac{- {s}^{2}}{{ω}_{H}^{2} + {ω}_{SA}^{2}}} + \frac{{( 1 - {e}^{- {μ}_{s} z} )}^{2} {ω}_{H}^{2}}{( 1 + {μ}_{a} Δ {z}_{D} ) {ω}_{SA}^{2}} {e}^{\frac{- {s}^{2}}{2 {ω}_{SA}^{2}}} ] , \qquad ( 2 )$$

where μₐ is the absorption coefficient of the tissue, ω_{H} and ω_{SA} are the the 1/e intensity radii in the absence and presence of absorption and scattering, respectively. The first term on the right side of Eq. 2, which is the single scattering component (Beer's law) is the contribution due to ballistically scattered light in the OCT signal measured in the detector plane. In OCT, ballistic light travels through the intervening turbid medium to and back from the tissue discontinuity in straight lines (i.e., without scattering). The third term represents the contribution due to multiply scattered light within the coherence time of the laser source, while the second term is a cross term related to the contribution due to both ballistic and multiple scattered return light. Note that the calculations here all consider an OCT system with dynamic focusing.

FIG. 6a indicates the relative contribution of single and multiply scattered light to the OCT signal with a separation of s=0. Already from FIG. 6a is quite clear that multiply scattered light dominates the OCT signal acquired from deeper in the sample. Indeed, the OCT signal is mainly composed of multiply scattered light, especially as the depth in the sample increases, contrary to the generally excepted premise that only ballistically scattered light contributes to image formation. The depth at which the multiply scattered light dominates the OCT signal depends on the scattering coefficient, as shown in FIG. 6b. As a consequence, variation the offset s between the illumination and collection paths allows for tuning of the relative contribution of single and multiply scattered light to the collected signal. An example of the heterodyne efficiency factor for various values of s is shown in FIG. 6c. At very short depths for ω_{H}=s, the heterodyne efficiency factor is highly suppressed, as expected, because the ballistic contribution to the OCT signal is proportional to

$${e}^{\frac{{D}^{2}}{2 {ω}_{H}^{2}}} = 1.$$

In contrast, for ω_{H}<s the heterodyne efficiency factor is closely approximated for that of s=0. As indicated in FIG. 6c for increasing depth z, Ψ coalesces with the (un-displaced) heterodyne efficiency for s=0 in the regime where multiple scattered light whose temporal dispersion is less than the coherence time of the laser source are the primary contribution to the OCT signal. Practically, this demonstrates that the optimization of s can lead to an optimal CNR for a selected depth. In the case where sensitivity is not a limiting factor, the dynamic range of the detector can be likewise optimized to a range relevant to the multiply scattered signals which have travelled deeper into the sample.

We acknowledge that the implementation where the illumination and collection paths are offset and parallel represents one case of a broader framework encompassing the spatial and angular offset between the illumination and collection paths. Thus, we can extend our model to also incorporate an angular offset and connect our framework to the dual axis scheme by incorporating a depth-dependent offset between the illumination and collection paths: s(z)=z tan α+s₀≈αz+s₀, where α is the angular offset and so is the lateral offset in the plane of the tissue discontinuity (typically the focal plane). Eqn. 1 incorporates both lateral and angular offset. The impact of incorporating an angular offset is shown in FIGS. 6c and d.

We note a key difference between spatially-offset OCT and dual-axis OCT. The angular relation between the illumination and collection paths in dual-axis OCT is designed to selectively collect light scattered ballistically at a particular angle to the illumination beam and is based on the principle that multiply scattered light, while collected by the coherence gate, contributes only an unwanted background signal that results on the degradation of OCT image contrast. Conversely, recent work modelling the OCT signal using the extended Huygens-Fresnel model has shown that multiply scattered light does indeed contain information about sample structure. As such, the spatial offset allows selective collection of the multiply scattered light, which enables deeper imaging since the multiply scattered light has typically travelled deeper into the sample. We acknowledge that using a spatial offset filters out the ballistic component, which leads to less light being collected for higher offset (signal loss). However, the specular reflections near the surface are also alleviated massively. As a result, the dynamic range of the image is decreased so that we can increase the exposure time to compensate the signal loss while obtaining a better contrast at depth. Additionally, since the offset can easily be tuned from s=0 to various offsets with s>0, images with and without the ballistic component can be acquired using the same OCT system. The magnitude of the spatial offset and the angular offset represent two factors that can be independently tuned based on the sample scattering properties in order to optimize the contrast at different regions by controlling the ratio of ballistically and multiply scattered light that are collected from different regions of the sample.

### Further Examples to Illustrate the Usefulness of the Present Disclosure

The examples below are just added for illustrative reasons and should in no way be considered at limiting the scope of protection.

### Assessment of Bone Quality:

Current assessment of bone quality relies on measurements of bone mineral density using dual-energy X-ray absorptiometry (DXA). However, DXA is an indirect measurement of bone quality and often underestimates fracture risk in patients with osteoporosis or diabetic bone disease. Additionally, DXA has insufficient resolution to segment the cortical and trabecular bone. Conventional OCT has sufficient resolution to enable this segmentation but is limited in its imaging depth due to the high scattering of bone tissue. The disclosure described here would enable the segmentation of bone layers and independent assessment of the material properties of each layer, which will provide a direct and localized measurement of bone quality.

### Identification and Classification of Tumors:

Tumors typically form in epithelial tissue, a type of tissue that forms a covering on all internal and external surface of the body. Depending on the organ, the consequential layer could be closer or further from the tissue surface. In any case, small variations in the local thickness of the epithelial tissues could be indicative of the formation of pre-cancerous lesions. The disclosure described here would improve the segmentation between layers and improve the detection of small changes in thickness. For slightly larger tumors, the disclosure could provide improved contrast between the tumor boundaries and the surrounding tissue. Additionally, as tumors grow, they tend to extend deeper into the tissue; the penetration depth of the tumor is an important component of classification/grading and observation of the margin is important for successful resection. The disclosure described here will improve the ability to detect the bottom of the tumor since it will improve segmentation deeper in tissue.

### Diagnosis of Retinal Diseases:

The retina is composed of 10 layers of different types of tissues. Local variation in the thickness of specific layers of the retina has been associated with various diseases including macular degeneration and Alzheimer's disease. The sensitivity with which these diseases can be detected early relies on the ability to detect very small changes in the thickness of the pertinent layers in the retina. Previous OCT-based approaches to improve segmentation of retinal layers have relied on improving the axial resolution through the adoption of light sources with increasingly larger bandwidths. Such light sources increase exponentially in price as the bandwidth is increased, and the other optical components required become significantly more expensive and difficult to fabricate. The disclosure described here provides an alternative approach to improving segmentation between retinal layers without relying on expensive, large bandwidth light sources.

Material Quality Assessment in Composite Materials and/or Polymers:

As in the biological examples, the disclosure described here can help to provide improved segmentation between regions in a material with different material and optical properties. This same approach could be used to identify local variations in material properties such as weaker sections of the material or areas with microcracks.

1. A method of using optical coherence tomography for training a machine learning model, the method comprising the steps of
illuminating a first sample having a known condition along an illumination path,
capturing a first training image of the sample along a first training collection path, wherein the first training collection path is spatially and/or angularly offset from the illumination path by a first training offset,
capturing a second training image of the sample along a second training collection path different from the first training collection path for providing a reference image, and
training a machine learning model based on the first and second training images and the known condition.

2. The method according to claim 1, wherein the first training image and the second training image are captured by an image capturing device, such as an image sensor or a camera, like a CCD camera, or by a photodetector, like a photodiode.

3. The method according to claim 1 or 2, wherein the method comprises the steps of
capturing a series of training images, wherein each of the training images of the series of training images has a training collection path different from the training collection paths of the other training images, wherein the series of training images comprises the first training image and the second training image, and
providing the series of training images as input to the model.

4. The method according to claim 3, wherein the series of training images is captured in one single exposure.

5. The method according to claim 3 or 4, wherein the method comprises the step of
moving the image capturing device in relation to the first sample from the first training collection path to the second training collection path for capturing the series of training images.

6. The method according to any of the preceding claims, wherein the method comprises the steps of
capturing a third training image of the sample along a third training collection path different from the first and second training collection paths for providing a second reference image,
optionally capturing a fourth training image of the sample along a fourth training collection path different from the first, second, and third training collection paths for providing a third reference image, and
training a machine learning model based on the first, second, and third training images, optionally also based on the fourth training image, and the known condition.

7. The method according to any of the preceding claims, wherein the machine learning model is a deep learning model or a neural network.

8. The method according to any of the preceding claims, wherein the known condition is the ground truth when training the model.

9. The method according to any of the preceding claims, wherein at least a first point in the first training image is correlated to at least a second point in the second training image, preferably wherein at least two first points in the first training image are correlated to at least two second points in the second training image.

10. The method according to according to any of the preceding claims, wherein the second training collection path is along the illumination path, or wherein the second training collection path is spatially and/or angularly offset from the illumination path by a second training offset different from the first training offset.

11. The method according to according to any of the preceding claims, wherein the first training offset is more than 5 μm, preferably more than 10 μm, most preferably more than 20 μm, and/or wherein the first training offset is less than 250 μm, preferably less than 200 μm, most preferably less than 150 μm.

12. The method according to according to any of the preceding claims, wherein the sample is illuminated by a light ray having a spot diameter on the sample, wherein the first training offset is at least half the spot diameter or at least the spot diameter, preferably more than two times the spot diameter, more preferably more than three times the spot diameter, even more preferably more than four times the spot diameter, and most preferably more than five times the spot diameter, and/or wherein the first training offset is less than 50 times the spot diameter, preferably less than 30 times the spot diameter, more preferably less than ten times the spot diameter, and most preferably less than eight times the spot diameter.

13. The method according to according to any of the preceding claims, wherein the method comprising steps of
capturing a plurality of images of the sample along a plurality of different training collection paths, wherein the plurality of different training collection paths are spatially and/or angularly offset from the illumination path by a plurality of different training offsets, and
providing the plurality of images as input to the model.

14. The method according to any of the preceding claims, wherein the illumination path and the first training collection path form an angle between 5° and 90°, preferably between 10° and 70°, more preferably between 15° and 50°, and most preferably between 20° and 40°, and/or wherein the illumination path and the second training collection path form an angle between 5° and 90°, preferably between 10° and 70°, more preferably between 15° and 50°, and most preferably between 20° and 40°.

15. The method according to any of the preceding claims, wherein the optical coherence tomography image (cross-sectional tomogram) is achieved by an interferometer with two optical paths, wherein the first training image comprises a number of first training images for different optical path length differences between the two optical paths.

16. The method according to claim 15, wherein the first training image for training the machine learning model is a first cross-sectional tomogram based on the number of first training images.

17. The method according to claim 15 or 16, wherein the second training image comprises a number of second training images for different optical path length differences between the two optical paths.

18. The method according to claim 17, wherein the second training image for training the machine learning model is a second cross-sectional tomogram based on the number of second training images.

19. The method according to claim 15 or 16, wherein the second training image comprises a number of second training images for different optical path length differences between the two optical paths, wherein a fifth training image comprises a number of fifth training images for different optical path length differences between the two optical paths, wherein the second training image for training the machine learning model is a compound of a second cross-sectional tomogram based on the number of second training images and a fifth cross-sectional tomogram based on the number of fifth training images.

20. The method according to any of the preceding claims, wherein the step of illuminating the first sample is performed by a tunable radiation source having a narrow, instantaneous line width.

21. The method according to claim 20, wherein the narrow, instantaneous line width is swept from around a first wavelength to around a second wavelength different from the first wavelength.

22. The method according to any of the preceding claims, wherein the illumination path is swept over the first sample from a first position to a second position different from the first position.

23. The method according to any of the preceding claims, wherein the first training offset and/or the second training offset is/are constant at least partly during the scanning of the illumination path.

24. The method according to any of the preceding claims, wherein the first training offset is varied/moved spatially and/or angularly during the step of capturing the first training image of the sample along the first training collection path from a first training offset position to a second training offset position differently from the first training offset position.

25. The method according to any of the preceding claims, wherein the second training collection path is swept from a first training collection path position to a second training collection path position differently from the first training collection path position.

26. A method of determining an unknown condition, wherein the method comprises the steps of
illuminating a sample along an illumination path,
capturing a first image of the sample along a first collection path, wherein the first collection path is spatially and/or angularly offset from the illumination path by a first offset, and
capturing a second image of the sample along a second collection path different from the first collection path,
determining an unknown condition based on the first and second images using a machine learning model trained according to any of the preceding claims.

27. A method of determining segmentation in a sample, wherein the method comprises the steps of
illuminating a sample along an illumination path,
capturing a first image of the sample along a first collection path, wherein the first collection path is spatially and/or angularly offset from the illumination path by a first offset,
capturing a second image of the sample along a second collection path different from the first collection path, and
relating the first image and the second image to each other for determining segmentation in the sample.

28. The method according to claim 26 or 27, wherein the method comprises any of the features or combinations of features according to any of the preceding claims 2-25.

29. An optical coherence tomography system, wherein the system comprises
a light source for illuminating a sample along an illumination path,
at least one optical instrument for capturing a first image of the sample along a first collection path, wherein the first collection path is spatially and/or angularly offset from the illumination path by a first offset, and for capturing a second image of the sample along a second collection path different from the first collection path, and
a processor configured to determine an unknown condition based on the first image and the second image using a machine learning model trained according to any of the preceding claims 1-25.

30. The system according to claim 29, wherein the model is configured to provide a condition based on
a first image of the sample captured along a first collection path, wherein the first collection path is spatially and/or angularly offset from the illumination path by a first offset, and
a second image of the sample along a second collection path.

31. The system according to claim 29 or 30, wherein the system is configured to control the optical system to change the spatial and/or angular offset of the collection path in relation to the illumination path.
